(19) United States
(12) Patent Application Publication
(10) Pub. No.: US 2021/0000758 A1
(43) Pub. Date: Jan. 7, 2021

(54) TREATMENT OF OCULAR DISEASES WITH OPHTHALMIC TAPINAROF COMPOSITIONS

(71) Applicant: SOL-GEL TECHNOLOGIES LTD., Ness Ziona (IL)

(72) Inventors: Moshe ARKIN, Kfar Shmaryahu (IL); Marcel ZIGHELBOIM, Kiryat Motzkin (IL)

(73) Assignee: SOL-GEL TECHNOLOGIES LTD., Ness Ziona (IL)

(21) Appl. No.: 16/929,400

(22) Filed: Jul. 15, 2020

Related U.S. Application Data
(63) Continuation-in-part of application No. PCT/IL2020/050677, filed on Jun. 17, 2020.
(60) Provisional application No. 62/862,141, filed on Jun. 17, 2019.

(51) Int. Cl. A61K 31/05; A61K 9/107; A61P 27/02; A61K 9/00; A61K 9/08; A61K 9/06; A61K 45/06

(52) U.S. Cl. CPC A61K 31/05; A61K 9/107; A61P 27/02; A61K 45/06; A61K 9/08; A61K 9/06; A61K 9/0048

(57) ABSTRACT

The present invention relates to the treatment of an ocular inflammatory disease or an ocular degeneration disease by ophthalmic administration of a composition comprising tapinarof and optionally at least one additional active agent. The composition of the present invention is useful for the treatment, prevention and/or alleviation of the symptoms of an ocular inflammatory disease or an ocular degeneration disease selected from uveitis, vitritis, dry eye disease (DED), macular degeneration, idiopathic orbital inflammatory disease (IOD), chorioretinal inflammation, keratitis, age-related macular degeneration and combinations thereof.

### CROSS REFERENCE TO RELATED APPLICATIONS

This application is a Continuation-in-Part of PCT International Application No. PCT/IL2020/050677 filed 17 Jun. 2020 which claims the benefit of U.S. Ser. No. 62/862,141, filed on 17 Jun. 2019, which is incorporated in its entirety herein by reference.

### FIELD OF THE INVENTION

The present invention, in some embodiments thereof, relates to treatment of an ocular inflammatory disease or an ocular degeneration disease by ophthalmic administration to a subject in need thereof of a composition comprising tapinarof and optionally at least one additional active agent. The composition of this invention is useful for the treatment, prevention or amelioration of ocular inflammatory diseases or of ocular degeneration disease and their symptoms.

### BACKGROUND

Ocular inflammatory diseases and/or ocular degeneration diseases include uveitis, vitritis, dry eye disease (DED), macular degeneration, idiopathic orbital inflammatory disease (IOD), chorioretinal inflammation, keratitis and combinations thereof. Ocular autoimmune diseases have been described in a recent article by Bose T. et al, “Dry eye disease and uveitis”, Autoimmune Reviews, vol. 15, issue 12, December 2016, pp. 1181-1192.

While DED affects the ocular surface, uveitis involves inflammation of the inner eye.

Uveitis is the inflammation of the uvea, the pigmented layer between the inner retina and the outer fibrous layer composed of the sclera and cornea. According to the site of the inflammation, there are several types of uveitis: anterior uveitis, interior uveitis, posterior uveitis and pan-uveitis (inflammation of all the uvea layers). Uveitis affects literally millions of people around the globe, with women dramatically over represented, particularly those women who have entered menopause. It has the potential for serious ocular consequences, beginning with the formation of dry spots on the cornea, progressing to epithelial defects or “abrasions” which resist healing, and then in some instances causing ulceration of the cornea, sometimes with perforation. Present medication of uveitis includes topical steroids (prednisolone acetate), injectable triamcinolone acetate, topical atropine or homatropine and methotrexate.

Dry eye disease (DED), also known as dry eye syndrome (DES) or keratoconjunctivitis sicca (KCS), affects up to 70% of older people. Medication of DED includes mild topical steroids (like prednisolone), lubricating tear ointments, immune-suppressants (like ciclosporin) and lifitegrast, Lifitegrast (Xiidra) is a new immune mediator drop for management of dry eye shown to be effective. (“Dry Eye”—The Ocular Immunology and Uveitis Foundation—www.uveitis.org). Some of the above treatments exhibit unwanted side-effects.

The mainstay of treatment for dry eye syndrome through the years has been replacement of fluid through the use of artificial tears. And while this is an important approach to the treatment of dry eye, it is by no means the only or the most important approach. The use of anti-inflammatory and immunomodulatory agents by topical application also plays a role in many patients' dry eye care, since inflammation of the lacrimal gland has been shown to be present and to be hindering the normal function of that tear-producing gland in a significant proportion of patients with dry eye disease. Hence, topical Alrex (loteprednol etabonate, a weak corticosteroid) and topical Restasis (containing cyclosporin) have been shown to be beneficial in such patients.

Macular degeneration, especially age-related macular degeneration (AMD or ARMD), a disease in which inflammation plays a clear role, is the leading cause of blindness. With ageing populations in many countries, more than 20% might have the disorder. Advanced age-related macular degeneration, including neovascular age-related macular degeneration (wet) and geographic atrophy (late dry), is associated with substantial, progressive visual impairment. The neovascular form of the disease represents approximately 10 percent of the overall disease prevalence, but it is responsible for 90 percent of the severe vision loss. Neovascular age-related macular degeneration is characterized by choroidal neovascularization that invades the subretinal space, often leading to exudation and hemorrhage. If the condition is left untreated, damage to photoreceptors and loss of central vision usually result, and after several months to years, the vessels are largely replaced by a fibrovascular scar. Major risk factors include cigarette smoking, nutritional factors, cardiovascular diseases, and genetic markers, including genes regulating complement, lipid, angiogenic, and extracellular matrix pathways. While no medication is available for dry AMD, wet AMD is treated with VEGF inhibitors (ranibizumab, aflibercept or pegaptanib).

Idiopathic orbital inflammatory disease (IOD), also known as orbital pseudotumor, is a benign, non-granulomatous orbital inflammatory process characterized by extraocular orbital and adnexal inflammation. Corticosteroids are the main treatment of IOD. Other alternative or adjuvant treatment options are NSAIDs, cytotoxic drugs (chlorambucil, cyclophosphamide), corticosteroid-sparing immunosuppressants (methotrexate, cyclosporine, azathioprine) and TNF-α inhibitors.

Chorioretinal inflammation, also known as chorioretinitis, is an inflammation of the choroid and retina of the eye. It is a form of posterior uveitis. If only the choroid is inflamed, it is termed choroiditis. Chorioretinitis is usually treated with a combination of corticosteroids and antibiotics.

Keratitis is a condition in which the cornea becomes inflamed. There are several types of keratitis, classified according to the infective cause, environmental aethiology (exposure keratitis, photokeratitis), etc. The treatment depends on the cause of keratitis. Infectious keratitis is treated with antibacterials (levofloxacin, gatifloxacin, moxifloxacin or ofloxacin).

There is an unmet need for novel and effective methods of treatment of ocular inflammatory diseases, essentially devoid of side-effects.

### SUMMARY OF THE INVENTION

This invention provides a composition and a method of treatment of ocular inflammatory diseases or ocular degeneration disease by administration to a subject in need thereof an ophthalmic composition comprising from tapinarof and optionally at least one additional active agent and a carrier suitable for ophthalmic administration.

This invention provides a composition and a method of treatment of ocular inflammatory diseases or ocular degeneration disease by administration to a subject in need thereof a topical ophthalmic composition comprising from about 0.01% w/w to about 10.0% w/w tapinarof and a carrier suitable for ophthalmic administration.

Also provided is a topical ophthalmic composition and a method of treatment of ocular inflammatory diseases or ocular degeneration disease, the composition further comprising from about 0.01% to about 10.0% w/w at least one additional active agent selected from a weak corticosteroid (loteprednol etabonate, prednisolone acetate, triamcinolone acetate), an immune suppressant (cyclosporin), an immune mediator (lifitegrast), an antimuscarinic agent (atropine, homatropine), a VGF inhibitor (ranibizumab, aflibercept, pegaptanib), an NSAID, a cytotoxic drug (chlorambucil, cyclophosphamide), a corticosteroid-sparing immunosuppressant (methotrexate, azathioprine), a TNF-α inhibitor, an antibiotic (levofloxacin, gatifloxacin, moxifloxacin, ofloxacin) and combinations thereof.

The present invention also provides dosage forms and kits comprising the above compositions.

Tapinarof (3,5-dihydroxy-4-isopropyl-trans-stilbene) is a pharmaceutical active agent investigated for the treatment of atopic dermatitis, psoriasis and psoriatic disorders (Zang Y N, et al., Int J Clin Pharmacol Ther. 2016 February; 54(2):87-95). The 3,5-dihydroxy-4-isopropyl-stilbene is also known as benvitimod.

The compositions, dosage forms, kits, implants and methods of this invention are suitable for the treatment, prevention or alleviation of an ocular inflammatory disease or an ocular degeneration disease and exhibit synergistic and/or additive effects which allow reducing the amounts of the active agents in the compositions.

### DETAILED DESCRIPTION OF THE INVENTION

This invention provides a composition and a method of treatment of ocular inflammatory diseases or ocular degeneration disease by administration to a subject in need thereof an ophthalmic composition comprising tapinarof and optionally at least one additional active agent and a carrier suitable for ophthalmic administration. In another embodiment, the composition is a topical ophthalmic composition. In another embodiment, the composition is an intraocular injectable composition.

In one embodiment, the present invention provides compositions, implants, dosage forms, kits and articles of manufacture that include tapinarof, optionally in combination with at least one additional active agent, for the treatment of an ocular inflammatory disease and/or ocular degeneration disease. The compositions can be administered to a subject in need thereof using a variety of ophthalmic compositions. The preferred route is the topical ophthalmic route and the preferred formulations are the drops, the cream, the ointment, the foam and the solution (see Examples 1-3).

In one embodiment, the present invention provides methods for the treatment, prevention and/or amelioration of an ocular inflammatory disease and/or an ocular degeneration disease, comprising administering (a) a topical ophthalmic composition comprising from about 0.01% w/w to about 10% w/w tapinarof; or (b) an intraocular injectable composition comprising from about 3 mg/ml to about 80 mg/ml tapinarof.

In another embodiment, the topical ophthalmic composition comprises from 0.01% w/w to 0.5% w/w tapinarof. In another embodiment, the composition comprises from 0.5% w/w to 1% w/w tapinarof. In another embodiment, the composition comprises from 1% w/w to 1.5% w/w tapinarof. In another embodiment, the composition comprises from 1.5% w/w to 2% w/w tapinarof. In another embodiment, the composition comprises from 2% w/w to 5% w/w tapinarof. In another embodiment, the composition comprises from 5% w/w to 10% w/w tapinarof. Each possibility represents a separate embodiment of the present invention.

In another embodiment, the injectable composition comprises from about 3 mg/ml to about 80 mg/ml tapinarof. In another embodiment, the intraocular injectable composition comprises from 3 mg/ml to 10 mg/ml tapinarof. In another embodiment, the intraocular injectable composition comprises from 10 mg/ml to 30 mg/ml tapinarof. In another embodiment, the intraocular injectable composition comprises from 20 mg/ml to 50 mg/ml tapinarof. In another embodiment, the intraocular injectable composition comprises from 30 mg/ml to 60 mg/ml tapinarof. In another embodiment, intraocular injectable composition comprises from 50 mg/ml to 80 mg/ml tapinarof. In another embodiment, the intraocular injectable composition comprises from 3 mg/ml to 20 mg/ml tapinarof. In another embodiment, the intraocular injectable composition comprises from 10 mg/ml to 20 mg/ml tapinarof. Each possibility represents a separate embodiment of the present invention.

In some embodiments, the composition, implants, method, and/or kit of this invention comprise additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

A non-limiting example of weak corticosteroid includes loteprednol etabonate, prednisolone acetate, triamcinolone acetate or combination thereof. A non-limiting example of an immune suppressant includes cyclosporin. A non-limiting example of an immune mediator includes lifitegrast. A non-limiting example of an antimuscarinic agent includes atropine, homatropine or combination thereof. A non-limiting example of a VGF inhibitor includes ranibizumab, aflibercept, pegaptanib or combination thereof. A non-limiting example of an NSAID include nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone or a combination thereof. A non-limiting example of a TNF-α inhibitor includes adalimumab. A non-limiting example of a cytotoxic drug includes chlorambucil, cyclophosphamide or combination thereof. A non-limiting example of corticosteroid-sparing immunosuppressant include methotrexate, azathioprine or combination thereof. A non-limiting example of an antibiotic includes levofloxacin, gatifloxacin, moxifloxacin, ofloxacin, or combination thereof.

In one embodiment, the present invention also provides a topical ophthalmic combination composition comprising tapinarof and from about 0.01% w/w to about 10.0% w/w at least one additional active agent selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof and a carrier suitable for topical administration. Each possibility represents a separate embodiment of the present invention.

In another embodiment, the composition is a topical composition comprising from about 0.01% w/w to about 10.0% w/w tapinarof. In another embodiment, the topical composition comprises from 0.25% w/w to 0.5% tapinarof. In another embodiment, the topical composition comprises from 0.5% w/w to 1% tapinarof. In another embodiment, the topical composition comprises from 1% w/w to 1.5% tapinarof. In another embodiment, the topical composition comprises from 1.5% w/w to 2% tapinarof. In another embodiment, the topical composition comprises from 2% w/w to 5% tapinarof. In another embodiment, the topical composition comprises from 5% w/w to 10% tapinarof. Each possibility represents a separate embodiment of the present invention.

In another embodiment, the composition is an intraocular injectable composition comprising from about 3 mg/ml to 80 mg/ml tapinarof. In another embodiment, the intraocular injectable composition comprises from 3 mg/ml to 10 mg/ml tapinarof. In another embodiment, the intraocular injectable composition comprises from 10 mg/ml to 30 mg/ml tapinarof. In another embodiment, the intraocular injectable composition comprises from 20 mg/ml to 50 mg/ml tapinarof. In another embodiment, the intraocular injectable composition comprises from 30 mg/ml to 60 mg/ml tapinarof. In another embodiment, intraocular injectable composition comprises from 50 mg/ml to 80 mg/ml tapinarof. In another embodiment, the intraocular injectable composition comprises from 3 mg/ml to 20 mg/ml tapinarof. In another embodiment, the intraocular injectable composition comprises from 10 mg/ml to 20 mg/ml tapinarof. Each possibility represents a separate embodiment of the present invention.

In another embodiment, the intraocular injectable composition further comprises from 3 mg/ml to 80 mg/ml additional active agent. In another embodiment, the additional active agent is selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof and a carrier suitable for intraocular injectable administration. In another embodiment, the composition comprises from 3 mg/ml to 10 mg/ml additional active agent. In another embodiment, the composition comprises from 5 mg/ml to 15 mg/ml additional active agent. In another embodiment, the composition comprises from 10 mg/ml to 20 mg/ml additional active agent. In another embodiment, the composition comprises from 20 mg/ml to 30 mg/ml additional active agent. In another embodiment, the composition comprises from 30 mg/ml to 50 mg/ml additional active agent. In another embodiment, the composition comprises from 40 mg/ml to 80 mg/ml additional active agent. In another embodiment, the composition comprises from 10 mg/ml to 30 mg/ml additional active agent. Each possibility represents a separate embodiment of the present invention.

In some embodiments, tapinarof and optionally at least one additional active agent in the compositions of this invention are included in an amount effective for treating, preventing or reducing the symptoms of an ocular inflammatory disease and/or ocular degeneration disease. The selection of the at least one additional active agent depends on the specific ocular inflammatory disease and/or ocular degeneration disease and the medical indication of the specific tapinarof/additional active agent combination. The concentrations of the active agents in the composition will depend on absorption, inactivation, excretion rates of the active compound, the synergistic or additive effects, the dosage schedule, and amount administered as well as other factors known to those of skill in the art. Generally, the dosages and concentrations will be lower, typically at least about or at 5 to 10% lower but up to about 15, 20, 25, 30, 35, 40, 50, 90 or 95% lower than the amount of tapinarof and optionally at least one additional active agent in the marketed single drug currently administered or considered for the treatment of an ocular inflammatory disease and/or an ocular degeneration disease. The dosage and regimen of administration may be determined by dose finding studies, as known in the art. Each possibility represents a separate embodiment of the present invention.

Exemplary dosages, strengths and concentrations of tapinarof in the tapinarof compositions administered topically, can be in the range of from about or at 0.1%, 0.25%, 0.5%, 1%, 2%, 3%, 4%, 5%, 6%, 7%, 8%, 9% or 10% w/w. Typical strengths in the topical combination compositions of this invention are 0.01%, 0.1%, 0.25%, 0.5%, 1% or 2% w/w tapinarof. The frequency of administration can be determined empirically. Exemplary frequencies are once daily, twice daily, three times per day, or four times per day, weekly, bi-weekly or monthly. Typical administration frequencies of the topical combination compositions of this invention are once daily and twice daily.

Exemplary dosages, strengths and concentrations of tapinarof in the intraocular injectable composition, can be in the range of from about or at 3 mg/ml, 5 mg/ml, 10 mg/ml, 15 mg/ml, 20 mg/ml, 25 mg/ml, 30 mg/ml, 40 mg/ml, 50 mg/ml, 60 mg/ml, 70 mg/ml or 80 mg/ml. Typical strengths in the combination compositions of this invention are 03 mg/ml, 5 mg/ml, 10 mg/ml, 15 mg/ml, 20 mg/ml, 25 mg/ml, 30 mg/ml, 40 mg/ml or 50 mg/ml tapinarof. The frequency of administration can be determined empirically. Exemplary frequencies are once daily, twice weekly, once weekly, twice monthly, monthly, every four to six weeks for a period of 4, 6, 8 or 12 months.

Dosage frequencies can be gradually attenuated over time and maintained at a steady dose suitable for long-term—six months, 1 year, 5 years, 10 years or more, up to lifelong administration to control the symptoms of the ocular inflammatory disease and/or of the ocular degeneration disease. For example, dosage administration can begin at from twice a day, to once a day, to two times a week, to once a week, to once every two weeks or less frequent than once every two weeks.

Pharmaceutical carriers or vehicles suitable for preparation of the topical ophthalmic compositions provided herein include any such carriers known to those skilled in the art to be suitable for the particular mode of administration.

Pharmaceutical carriers or vehicles suitable for preparation of intraocular injectable compositions provided herein include any such carriers known to those skilled in the art to be suitable for the particular mode of administration.

The resulting topical composition may be a lotion, a solution, a suspension, an emulsion or the like and is formulated as drops, creams, gels, ointments, emulsions, solutions, elixirs, lotions, suspensions, tinctures, pastes, foams, aerosols, sprays, foams, or any other formulation suitable for topical ophthalmic administration. The preferred topical compositions are the drops, the cream, the ointment, the solution, the foam and the lotion. In another embodiment, the topical composition includes nanomicelles, nanospheres or nanoparticles comprising an active agent (e.g. tapinarof).

In some embodiments, the topical composition is an aqueous ophthalmic solution, wherein the solution comprises nanomicelles.

In some embodiments, the topical composition is an ophthalmic solution, wherein the solution comprises nanomicelles, nanospheres or nanoparticles of an active agent (tapinarof).

In some embodiments, the topical composition is an ophthalmic solution, wherein the solution comprises nanomicelles, nanospheres or nanoparticles comprising tapinarof and optionally at least additional one active agent. In another embodiment, the tapinarof is loaded within the nanoparticles or nanospheres.

Pharmaceutical carriers or vehicles suitable for administration of the active agents provided herein include any such carriers known to those skilled in the art to be suitable for the particular mode of administration. In addition, the active agent tapinarof may be formulated as the sole pharmaceutically active agent in the composition or may be combined with at least one additional active agent. The active agents are included in the carrier in an amount sufficient to exert a therapeutically useful effect i.e., amelioration of the symptoms of the ocular inflammatory disease and/or of the ocular degeneration disease, with minimal or no toxicity or other side effects. Generally, emollient or lubricating vehicles that help hydrate the eye are more preferred than volatile vehicles, such as ethanol, that dry the eye. Exemplary ingredients or vehicles for preparing compositions for use with topical ophthalmic compositions, (all within the regulatorily approved concentration ranges) are petrolatum, light mineral oil, lanolin, white wax, PEG 400, glycerin and cellulose derivatives. Suitable vehicles for intraocular injectable compositions of this invention are water or water and saline solutions.

Suitable pharmaceutically and dermatologically acceptable vehicles for topical application include those suited for use in drops, lotions, creams, ointments, solutions, gels, foams and the like. Generally, the vehicle is either organic in nature or an aqueous emulsion and capable of including the selected compound or compounds, which may be micronized, dispersed, suspended or dissolved therein. The vehicle may include pharmaceutically-acceptable emollients, moisturizers, penetration enhancers, fragrances and emulsifiers. In another embodiment, the topical composition includes nanomicelles, nanospheres or nanoparticles comprising an active agent (e.g. tapinarof). In another embodiment, the tapinarof and optionally the additional active agent are loaded within the nanoparticles or nanospheres, wherein the nanoparticles or nanospheres may be used as a drug carrier.

The compositions according to the invention are pharmaceutical compositions, and especially topical ophthalmic compositions, which may be in any form conventionally used for topical ophthalmic application. By addition of a fatty or oily phase, they may also be in the form of dispersions of the lotion or serum type, emulsions of liquid or semi-liquid consistency obtained by dispersing a fatty phase in an aqueous phase (O/W) or conversely (W/O), or suspensions or emulsions of soft, semi-liquid or solid consistency of the cream, gel or ointment type, or alternatively multiple emulsions (W/O/W or O/W/O), microemulsions, microparticles or vesicular dispersions of ionic and/or nonionic type, or wax/aqueous phase dispersions.

In some embodiments, said water in said oil in water emulsion further comprises at least one water soluble humectant.

In other embodiments, said at least one water soluble humectant is selected from the group consisting of propylene glycol, glycerin, and polyethylene glycol-X, where X is in the range of 200 to 10,000. Each possibility represents a separate embodiment of the present invention.

In further embodiments, there is provided a composition of the invention comprising, as a single pharmaceutical active agent, tapinarof for topical ophthalmic use or formulated for intraocular injection in the treatment of an ocular inflammatory disease and/or of an ocular degeneration disease. In some embodiments, the composition of the present invention comprises tapinarof and at least one additional active agent, wherein said additional active agent is selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof.

In further embodiments, the compositions of this invention for treating the ocular inflammatory disease and/or the ocular degeneration disease are controlled or slowed release drug delivery system, wherein the active agent is encapsulated, coated, adsorbed, embedded, impregnated, dispersed, entrapped, or encased in a polymeric material and providing a sustained release formulation. In another embodiment, the active agent(s) are encapsulated in a liposome. In another embodiment, the active agent is encapsulated in nanomicelles, or the active agent is loaded in nanoparticles or nanospheres. In another embodiment, the tapinarof and optionally the additional active agent are loaded within the nanoparticles or nanospheres, wherein the nanoparticles or nanospheres may be used as a drug carrier.

In other embodiments, microcapsules are formed by the encapsulation process disclosed in the following publications (herein incorporated by reference): U.S. Pat. Nos. 7,629,394, 9,205,395, US 2015/0328615, US 2014/0186630. Controlled release microcapsules: IN01958CH2007, IN02080CH2007, U.S. Pat. Nos. 4,235,872, 4,670,250, EP 0248531, U.S. Pat. Nos. 4,970,031, 5,238,714, WO9321764, U.S. Pat. No. 5,575,987, WO9420075, US 2004/137031, US 2006/003014, US 2010/180464.

When referring to a “controlled or slowed release drug delivery system” it should be understood to relate to a delivery system (which in the present invention is a topical delivery system) that enables the release of the pharmaceutical active agent in predetermined amounts over a specified period.

### Methods of Treatment

According to an aspect of the invention, there is provided a method of treatment of an ocular inflammatory disease and/or an ocular degeneration disease, comprising administering to a subject in need thereof a therapeutically effective amount of an ophthalmic composition comprising tapinarof and optionally at least one additional active agent. In another embodiment, the composition is a topical ophthalmic composition. In another embodiment, the composition is an intraocular injectable composition.

In some embodiments, the therapeutically effective amount is an effective amount of tapinarof and optionally at least one additional active agent, namely an amount which will cure, treat, mitigate and/or prevent the ocular inflammatory disease and/or the ocular degeneration disease.

In some embodiments, co-administration of tapinarof and at least one additional active agent in one composition or two separate compositions administered sequentially or simultaneously, in either order, exhibits an additive and/or synergistic effect while treating, preventing or alleviating ocular inflammatory disease and/or ocular degeneration disease.

In some other embodiments, the co-administration may be made either by administration of a single combination composition, or alternatively by separate administration of a first composition comprising tapinarof and a second composition comprising at least one additional active agent.

Regimen of Administration of Ophthalmic Tapinarof Compositions for Treatment of an Ocular Inflammatory Disease and/or an Ocular Degeneration Disease

Therapeutically effective concentrations of tapinarof and optionally at least one additional active agent in the compositions of this invention for treatment, prevention or amelioration of the symptoms manifested by the ocular inflammatory disease and/or the ocular degeneration disease are determined by empirical methods known in the art.

The concentration of the additional active agents in the composition will depend on absorption, inactivation, excretion rates of the active compound, synergistic and/or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of tapinarof and at least one additional active agent in the developed or marketed single drug currently being developed or used for the treatment of the ocular inflammatory disease and/or the ocular degeneration disease. The dosage and regimen of administration may be determined by dose finding studies, as known in the art.

Exemplary dosages, strengths and concentrations of tapinarof in the tapinarof compositions administered topically, can be in the range of from about 0.01%, 0.1%, 0.25%, 0.5%, 1%, 2%, 3%, 4%, 5%, 6%, 7%, 8%, 9% or 10% w/w.

Typical tapinarof strengths in the topical combination compositions of this invention are 0.01%, 0.1%, 0.25%, 0.5%, 1%, 2%, 5% or 10% w/w tapinarof.

The frequency of administration can be determined empirically. Exemplary frequencies are once daily, twice daily, weekly, bi-weekly or monthly.

Typical administration frequencies of the topical combination compositions of this invention are once daily and twice daily.

Exemplary dosages, strengths and concentrations of tapinarof in the intraocular injectable composition, can be in the range of from about or at 3 mg/ml, 5 mg/ml, 10 mg/ml, 15 mg/ml, 20 mg/ml, 25 mg/ml, 30 mg/ml, 40 mg/ml, 50 mg/ml, 60 mg/ml, 70 mg/ml or 80 mg/ml. Typical strengths in the combination compositions of this invention are 03 mg/ml, 5 mg/ml, 10 mg/ml, 15 mg/ml, 20 mg/ml, 25 mg/ml, 30 mg/ml, 40 mg/ml or 50 mg/ml tapinarof. The frequency of administration can be determined empirically. Exemplary frequencies are once daily, twice weekly, once weekly, twice monthly, monthly, every four to six weeks for a period of 4, 6, 8 or 12 months.

Dosage frequencies can be gradually attenuated over time and maintained at a steady dose suitable for long-term—six months, 1 year, 5 years, 10 years or more, up to lifelong administration to control the symptoms of an ocular inflammatory disease and/or of an ocular degeneration disease. For example, dosage administration can begin at from twice a day, to once a day, to two times a week, to once a week, to once every two weeks or less frequent than once every two weeks.

### Kits

There are provided kits containing the dosage forms and compositions of this invention, optionally including instructions for administration.

The compositions provided herein can be packaged as articles of manufacture containing packaging material, a composition provided herein, and a label that indicates that the composition is for treating an ocular inflammatory disease and/or an ocular degeneration disease, and is formulated for topical ophthalmic delivery.

The articles of manufacture provided herein contain packaging materials. Packaging materials for use in packaging pharmaceutical products are well known to those of skill in the art. Examples of pharmaceutical packaging materials include, but are not limited to drop dispensers, bottles, tubes, containers, application syringes and any packaging material suitable for the selected formulation and intended mode of administration and treatment.

### Implant

In some embodiments this invention provides an implant comprising tapinarof for use in treating ocular inflammatory disease and/or an ocular degeneration disease.

In some embodiments this invention provides an implant comprising tapinarof for use in treating age-related macular degeneration.

In another embodiment, the implant is implanted in the retina of the eye. In another embodiment, the implant comprises a layer of a composition comprising tapinarof and optionally with at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

In some embodiments this invention provides an implant comprising from about 0.5 mg to about 80 mg tapinarof. In some embodiments this invention provides an implant comprising from about 0.5 mg to about 80 mg tapinarof and optionally from about 0.5 mg to 10 mg additional active agent.

In another embodiment, the implant is a plastic rod with a pellet comprising tapinarof (or optionally with at least one additional active agent) on the end. The pellet slowly dissolves and releases the medication into the fluid in the eye. In another embodiment, the implant can release the medicine (Tapinarof) for 2 to 4 years.

### Embodiments

In some embodiments, there is provided an ophthalmic composition for the treatment, prevention and/or amelioration of an ocular inflammatory disease and/or an ocular degeneration disease, comprising from about 0.01% w/w to about 10.0% w/w tapinarof and a carrier suitable for topical ophthalmic administration or from about 3 mg/ml to about 80 mg/ml tapinarof and a carrier suitable for intraocular injectable administration.

In some embodiments, there is provided a topical ophthalmic composition for the treatment, prevention and/or amelioration of an ocular inflammatory disease and/or an ocular degeneration disease, comprising from about 0.01% w/w to about 10.0% w/w tapinarof and a carrier suitable for topical ophthalmic administration.

In some embodiments, there is provided an intraocular injectable composition for the treatment, prevention and/or amelioration of an ocular inflammatory disease and/or an ocular degeneration disease, comprising from about 3 mg/ml to about 80 mg/ml tapinarof and a carrier suitable for intraocular injectable administration.

In some other embodiments, there is provided a topical ophthalmic composition for the treatment, prevention and/or amelioration of an ocular inflammatory disease and/or an ocular degeneration disease, comprising from about 0.01% w/w to about 10.0% w/w tapinarof and a carrier suitable for topical ophthalmic administration, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof.

In some other embodiments, there is provided an intraocular injectable composition for the treatment, prevention and/or amelioration of an ocular inflammatory disease and/or an ocular degeneration disease, comprising from about 3 mg/ml to about 80 mg/ml tapinarof and a carrier suitable for intraocular injectable administration, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof.

According to some embodiments, there is provided a topical ophthalmic composition for the treatment, prevention and/or amelioration of an ocular inflammatory disease and/or an ocular degeneration disease, comprising from about 0.01% w/w to about 10.0% w/w tapinarof and a carrier suitable for topical ophthalmic administration, further comprising from about 0.01% w/ to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

According to some embodiments, there is provided an intraocular injectable composition for the treatment, prevention and/or amelioration of an ocular inflammatory disease and/or an ocular degeneration disease, comprising from about 3 mg/ml to about 80 mg/ml tapinarof and a carrier suitable for intraocular injectable administration, further comprising from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

According to some embodiments, there is provided an intraocular injectable composition for the treatment, prevention and/or amelioration of an ocular inflammatory disease and/or an ocular degeneration disease, comprising from about 3 mg/ml to about 80 mg/ml tapinarof and a carrier suitable for intraocular injectable administration, further comprising from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof; wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof.

According to some embodiments, there is provided an intraocular injectable composition for the treatment, prevention and/or amelioration of age-related macular degeneration (AMD), comprising from about 3 mg/ml to about 80 mg/ml tapinarof and a carrier suitable for intraocular injectable administration. In another embodiment, the age-related macular degeneration is neovascular age-related macular degeneration (wet). In another embodiment, the age-related macular degeneration is geographic atrophy (late dry).

According to some embodiments, there is provided an intraocular injectable composition for the treatment, prevention and/or amelioration of age-related macular degeneration (AMD), comprising from about 3 mg/ml to about 80 mg/ml tapinarof and further comprising from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

According to some other embodiments, there is provided a topical ophthalmic composition for the treatment, prevention and/or amelioration of an ocular inflammatory disease and/or an ocular degeneration disease, comprising from about 0.01% w/w to about 10.0% w/w tapinarof and a carrier suitable for topical ophthalmic administration, further comprising from about 0.01% w/ to about 10% w/w at least one additional active agent selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof.

According to some other embodiments, there is provided an intraocular injectable composition for the treatment, prevention and/or amelioration of an ocular inflammatory disease and/or an ocular degeneration disease, comprising from about 3 mg/ml to about 80 mg/ml tapinarof and a carrier suitable for intraocular injectable administration, further comprising from about 3 mg/ml to about 80 mg/ml at least one additional active agent selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof.

In some embodiments, there is provided a dosage form for the treatment, prevention and/or amelioration of an ocular inflammatory disease and/or an ocular degeneration disease, comprising from about 0.01% w/w to about 10.0% w/w tapinarof and a carrier suitable for topical ophthalmic administration, wherein the dosage form is selected from an ointment, a suspension, a cream, a spray, a lotion, a gel, an emulsion, a solution (including nanomicellar solutions), an elixir, a tincture, a paste, a foam and drops. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a dosage form for the treatment, prevention and/or amelioration of an ocular inflammatory disease and/or an ocular degeneration disease, comprising from about 0.01% w/w to about 10.0% w/w tapinarof and a carrier suitable for topical ophthalmic administration, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof, and wherein the dosage form is selected from an ointment, a suspension, a cream, a spray, a lotion, a gel, an emulsion, a solution (including nanomicellar solutions), an elixir, a tincture, a paste, a foam and drops. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a dosage form for the treatment, prevention and/or amelioration of an ocular inflammatory disease and/or an ocular degeneration disease, comprising from about 0.01% w/w to about 10.0% w/w tapinarof and a carrier suitable for topical ophthalmic administration, and optionally from about 0.01% w/ to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the dosage form is selected from an ointment, a suspension, a cream, a spray, a lotion, a gel, an emulsion, a solution, an elixir, a tincture, a paste, a foam and drops. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a dosage form for the treatment, prevention and/or amelioration of an ocular inflammatory disease and/or an ocular degeneration disease, comprising from about 0.01% w/w to about 10.0% w/w tapinarof and a carrier suitable for topical ophthalmic administration, and optionally from about 0.01% w/ to about 10% w/w at least one additional active agent selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof, wherein the dosage form is selected from an ointment, a suspension, a cream, a spray, a lotion, a gel, an emulsion, a solution (including nanomicellar solutions), an elixir, a tincture, a paste, a foam and drops. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a method of treatment, prevention and/or alleviation of an ocular inflammatory disease and/or an ocular degeneration disease, comprising topically administering to the eyes of a subject in need thereof a therapeutically effective amount of a composition comprising from about 0.01% w/w to about 10.0% w/w tapinarof and a carrier suitable for topical ophthalmic administration, wherein the composition is formulated as an ophthalmic ointment, suspension, a spray, a lotion, a gel, emulsion, solution (including nanomicellar solutions), an elixir, a tincture, a paste, drops, cream or foam. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a method of treatment, prevention and/or alleviation of an ocular inflammatory disease and/or an ocular degeneration disease, comprising administering to the eyes of a subject in need thereof a therapeutically effective amount of an intraocular injectable composition comprising from about 3 mg/ml to about 80 mg/ml tapinarof and a carrier suitable for intraocular injectable administration.

In some embodiments, there is provided a method of treatment, prevention and/or alleviation of an ocular inflammatory disease and/or an ocular degeneration disease, comprising topically administering to the eyes of a subject in need thereof a therapeutically effective amount of a composition comprising from about 0.01% w/w to about 10.0% w/w tapinarof and a carrier suitable for topical ophthalmic administration, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof, and wherein the composition is formulated as an ophthalmic ointment, suspension, a spray, a lotion, a gel, emulsion, solution (including nanomicellar solutions), an elixir, a tincture, a paste, drops, cream or foam. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a method of treatment, prevention and/or alleviation of an ocular inflammatory disease and/or an ocular degeneration disease comprising administering to the eyes of a subject in need thereof a therapeutically effective amount of an intraocular injectable composition comprising from about 3 mg/ml to about 80 mg/ml tapinarof and a carrier suitable for intraocular injectable administration, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof.

In some embodiments, there is provided a method of treatment, prevention and/or alleviation of age-related macular degeneration (AMD), comprising administering to the eyes of a subject in need thereof a therapeutically effective amount of an intraocular injectable composition comprising from about 3 mg/ml to about 80 mg/ml tapinarof and a carrier suitable for intraocular injectable administration. In another embodiment, the age-related macular degeneration is neovascular age-related macular degeneration (wet). In another embodiment, the age-related macular degeneration is geographic atrophy (late dry).

In some embodiments, there is provided a method of treatment, prevention and/or alleviation of an ocular inflammatory disease and/or an ocular degeneration disease, comprising topically administering to the eyes of a subject in need thereof a therapeutically effective amount of a composition comprising from about 0.01% w/w to about 10.0% w/w tapinarof and a carrier suitable for topical ophthalmic administration, and optionally from about 0.01% w/ to about 10% w/w at least one additional active agent selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof and wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof, and wherein the composition is formulated as an ophthalmic ointment, suspension, a spray, a lotion, a gel, emulsion, solution (including nanomicellar solutions), an elixir, a tincture, a paste, drops, cream or foam. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a method of treatment, prevention and/or alleviation of an ocular inflammatory disease and/or an ocular degeneration disease, comprising administering to the eyes of a subject in need thereof a therapeutically effective amount of an intraocular injectable composition comprising from about 3 mg/ml to about 80 mg/ml tapinarof and a carrier suitable for intraocular injection, and optionally from about 3 mg/ml to about 80 mg/ml at least one additional active agent selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof and wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof.

In some embodiments, there is provided a method of treatment, prevention and/or alleviation of an ocular inflammatory disease and/or an ocular degeneration disease, comprising topically administering to the eyes of a subject in need thereof a therapeutically effective amount of a composition comprising from about 0.01% w/w to about 10.0% w/w tapinarof and a carrier suitable for topical ophthalmic administration and optionally from about 0.01% w/ to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the composition is formulated as an ophthalmic ointment, suspension, a spray, a lotion, a gel, emulsion, solution (including nanomicellar solutions), an elixir, a tincture, a paste, drops, cream or foam. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a method of treatment, prevention and/or alleviation of an ocular inflammatory disease and/or an ocular degeneration disease, comprising administering to the eyes of a subject in need thereof a therapeutically effective amount of intraocular injectable composition comprising from about 3 mg/ml to about 80 mg/ml tapinarof and a carrier suitable for intraocular injectable administration, and optionally from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

In some embodiments, there is provided a method of treatment, prevention and/or alleviation of an ocular inflammatory disease and/or an ocular degeneration disease, comprising topically administering to the eyes of a subject in need thereof a therapeutically effective amount of a composition comprising from about 0.01% w/w to about 10.0% w/w tapinarof and a carrier suitable for topical ophthalmic administration and optionally from about 0.01% w/ to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the ocular inflammatory disease is selected from uveitis, vitritis, dry eye disease (DED), macular degeneration, idiopathic orbital inflammatory disease (IOD), chorioretinal inflammation, keratitis and combinations thereof, and wherein the composition is formulated as an ophthalmic ointment, suspension, a spray, a lotion, a gel, emulsion, solution (including nanomicellar solutions), an elixir, a tincture, a paste, drops, cream or foam. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a method of treatment, prevention and/or alleviation of an ocular inflammatory disease and/or an ocular degeneration disease, comprising administering to the eyes of a subject in need thereof a therapeutically effective amount of an intraocular injectable composition comprising from about 3 mg/ml to about 80 mg/ml tapinarof and a carrier suitable for intraocular injectable administration, and optionally from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, and wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof.

In some other embodiments, there is provided a method of treatment, prevention and/or alleviation of an ocular inflammatory disease and/or an ocular degeneration disease, comprising topically administering to the eyes of a subject in need thereof a therapeutically effective amount of a composition comprising from about 0.01% w/w to about 10.0% w/w tapinarof and a carrier suitable for topical ophthalmic administration and optionally from about 0.01% w/ to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof.

According to some embodiments, there is provided a method of treatment, prevention and/or alleviation of an ocular inflammatory disease and/or an ocular degeneration disease, wherein the treatment comprises once daily or twice daily topical administration to a subject in need thereof of a therapeutically effective amount of a composition comprising from about 0.01% w/w to about 10.0% w/w tapinarof and a carrier suitable for topical ophthalmic administration and optionally from about 0.01% w/ to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof.

According to some embodiments, there is provided a method of treatment, prevention and/or alleviation of an ocular inflammatory disease and/or an ocular degeneration disease, wherein the treatment comprises once daily or twice daily topical administration to a subject in need thereof of a therapeutically effective amount of a composition comprising from about 0.01% w/w to about 10.0% w/w tapinarof and a carrier suitable for topical ophthalmic administration. In another embodiment, the ocular inflammatory disease is selected and/or the ocular degeneration disease from uveitis, vitritis, dry eye disease (DED), macular degeneration, idiopathic orbital inflammatory disease (IOD), chorioretinal inflammation, keratitis, age-related macular degeneration (AMD) and combinations thereof.

According to some embodiments, there is provided a method of treatment, prevention and/or alleviation of an ocular inflammatory disease and/or an ocular degeneration disease, wherein the treatment comprises administering an intraocular injectable composition to a subject in need thereof comprising from about 3 mg/ml to about 80 mg/ml tapinarof and a carrier suitable for intraocular injectable composition. In another embodiment, the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof.

According to some other embodiments, there is provided a method of treatment, prevention and/or alleviation of an ocular inflammatory disease and/or an ocular degeneration disease, wherein the treatment comprises once daily or twice daily topical administration to a subject in need thereof of a therapeutically effective amount of a composition comprising from about 0.01% w/w to about 10.0% w/w tapinarof and a carrier suitable for topical ophthalmic administration and from about 0.01% w/ to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof. In another embodiment, the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof.

According to some embodiments, there is provided a method of treatment, prevention and/or alleviation of an ocular inflammatory disease and/or an ocular degeneration disease, wherein the treatment comprises administering an intraocular injectable composition to a subject in need thereof comprising from about 3 mg/ml to about 80 mg/ml tapinarof and a carrier suitable for intraocular injectable administration and from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof. In another embodiment, the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, age-related macular degeneration (AMD) and combinations thereof.

In some embodiments, there is provided a method of treatment, prevention and/or alleviation of an ocular inflammatory disease and/or an ocular degeneration disease, wherein the treatment comprises once daily or twice daily topical administration to a subject in need thereof of a therapeutically effective amount of a composition comprising from about 0.01% w/w to about 10.0% w/w tapinarof and a carrier suitable for topical ophthalmic administration and optionally from about 0.01% w/ to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein tapinarof and the at least one additional active agent exhibit an additive or synergistic effect, thereby allowing to reduce the amounts of the active agents in the composition.

In some embodiments, there is provided a method of treatment, prevention and/or alleviation of an ocular inflammatory disease and/or an ocular degeneration disease, wherein the treatment comprises to a subject in need thereof a therapeutically effective amount of an intraocular injectable composition comprising from about 3 mg/ml to about 80 mg/ml tapinarof and a carrier suitable for intraocular injectable administration and optionally from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein tapinarof and the at least one additional active agent exhibit an additive or synergistic effect, thereby allowing to reduce the amounts of the active agents in the composition.

In some other embodiments, there is provided a regimen of administration comprising the once daily or twice daily administration to the eyes of a patient in need thereof of a therapeutically effective amount of a topical ophthalmic composition for the treatment, prevention and/or amelioration of an ocular inflammatory disease and/or an ocular degeneration disease, comprising from about 0.01% w/w to about 10.0% w/w tapinarof and a carrier suitable for topical ophthalmic administration until remission or alleviation of the ocular inflammatory disease and/or the ocular degeneration disease symptoms.

In some other embodiments, there is provided a regimen of administration comprising the once daily, twice weekly, once weekly, twice monthly, monthly or every four to six weeks administration to the eyes of a patient in need thereof of a therapeutically effective amount of an intraocular injectable composition for the treatment, prevention and/or amelioration of an ocular inflammatory disease and/or an ocular degeneration disease, comprising from about 3 mg/ml to about 80 mg/ml tapinarof and a carrier suitable for intraocular injection until remission or alleviation of the ocular inflammatory disease and/or the ocular degeneration disease symptoms.

According to some embodiments, there is provided a regimen of administration comprising the once daily or twice daily administration to the eyes of a patient in need thereof of a therapeutically effective amount of a topical ophthalmic composition for the treatment, prevention and/or amelioration of an ocular inflammatory disease and/or an ocular degeneration disease, comprising from about 0.01% w/w to about 10.0% w/w tapinarof and a carrier suitable for topical ophthalmic administration and from about 0.01% w/ to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, until remission or alleviation of the ocular inflammatory disease and/or the ocular degeneration disease symptoms. In another embodiment, the at least one additional active agent is selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof.

According to some embodiments, there is provided a regimen of administration comprising administering once daily, twice weekly, once weekly, twice monthly, monthly, every four to six weeks administration to the eyes of a patient in need thereof a therapeutically effective amount of an intraocular injectable composition for the treatment, prevention and/or amelioration of an ocular inflammatory disease and/or an ocular degeneration disease, comprising from about 3 mg/ml to about 80 mg/ml tapinarof and a carrier suitable for intraocular injectable administration and from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, until remission or alleviation of the ocular inflammatory disease and/or the ocular degeneration disease symptoms. In another embodiment, the at least one additional active agent is selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof.

According to some other embodiments, there is provided a regimen of administration, comprising the once daily or twice daily administration to the eyes of a subject in need thereof a topical ophthalmic composition comprising from about 0.01% w/w to about 10.0% w/w tapinarof and a carrier suitable for topical ophthalmic administration and optionally from about 0.01% w/ to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis and combinations thereof.

According to some other embodiments, there is provided a regimen of administration, comprising the once daily, twice weekly, once weekly, twice monthly, monthly, every four to six weeks administration to the eyes of a subject in need thereof an intraocular injectable composition comprising from about 3 mg/ml to about 80 mg/ml tapinarof and a carrier suitable for intraocular injection and optionally from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis and combinations thereof.

In some embodiments, there is provided a kit comprising instructions for use and one or more dosage forms of this disclosure, comprising from about 0.01% w/w to about 10.0% w/w tapinarof and a carrier suitable for topical ophthalmic administration and optionally from about 0.01% w/ to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the composition is formulated as an ophthalmic ointment, suspension, solution, drops, cream or foam.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

As used herein, the term “micelle” or “nanomicelle” refer to an aggregate (or cluster) of surfactant molecules. In some embodiments, ophthalmic compositions of the present invention include an aqueous, clear or mixed micellar solution. Addition of water to a drug polymer mixture in organic solvent should spontaneously generate micelles thereby entrapping the pharmaceutical active agent in the hydrophobic core of mixed nanomicelles.

The term “Intraocular injection” refers herein to an injection into the eye. Non limiting examples include injection into the vitreous (intravitreous injection) or subretinal (interphotoreceptor) space; into the tissue surrounding the eye, or intraocular injection refers to periocular and periorbital injection.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader. All the information contained therein is incorporated herein by reference.

### Example 1

Preparation of a Tapinarof Ophthalmic Ointment Composition

The topical ophthalmic tapinarof ointment consists of:

0.01-10.0% w/w tapinarof,

50-65% w/w white petrolatum,

6% woolfat,

15-30% w/w liquid paraffin,

3-10% w/w PEG-4000 or PEG-400

0.5% phenylethyl alcohol

0.1-1.0% w/w alpha-tocopherol

The ointment composition is prepared by the following steps:
1. Weigh liquid paraffin in a bottle. Remove air bubbles in the liquid paraffin by applying vacuum for a period of 30 minutes.
2. Melt white petrolatum and wool fat on a stir plate at approximately 70° C.
3. Mix gently the liquid paraffin, white petrolatum and wool fat at approximately 70° C. for a period of 15 minutes or until the ingredients are uniformly mixed.
4. Weigh tapinarof having an average particle size of less than 1 μm and (partly) dissolve it in PEG 400 or PEG 4000 bp sonicating in a water bath maintained at 45° C.
5. Add alpha-tocopherol and phenylethyl alcohol to the PEG containing tapinarof.
6. Add (partly) dissolved tapinarof and mix for a period of 45 minutes or until a uniform dispersion is obtained.
7. Sterilize the tapinarof topical ophthalmic ointment by filtering the molten ointment through a 0.2-micron filter.
8. Fill the sterilized tapinarof ointment in a tube or other delivery system.

### Example 2

### Preparation of a Tapinarof Eye-Drop Suspension Composition

The topical eye drop tapinarof suspension composition consists of:

0.01-10.0% w/w tapinarof,

0.1-0.3% w/w edetate disodium,

0.2-1.0% w/w glycerin,

0.1-1% w/w povidone,

0.1-0.25% w/w tyloxapol,

0.01% benzalkonium chloride.

Add hydrochloric acid or sodium hydroxide to adjust the pH to 5.3-5.6.

The suspension is sterile and essentially isotonic.

### Example 3

### Preparation of a Tapinarof/Prednisolone Acetate Eye-Drop Suspension Composition

The topical eye drop tapinarof/prednisolone acetate suspension composition consists of:

0.01-10.0% w/w tapinarof,

1% w/w prednisolone acetate,

0.1-0.3% w/w edetate disodium,

0.2-1.0% w/w glycerin,

0.1-1% w/w povidone,

0.1-0.25% w/w tyloxapol,

0.01% benzalkonium chloride.

Add hydrochloric acid or sodium hydroxide to adjust the pH to 5.3-5.6.

The suspension is sterile and essentially isotonic.

1. A topical ophthalmic composition for the treatment, prevention and/or amelioration of an ocular inflammatory disease or ocular degeneration disease, comprising from about 0.01% w/w to about 10.0% w/w tapinarof and a carrier suitable for topical ophthalmic administration.

2. The composition of claim 1, wherein the ocular inflammatory disease or the ocular degeneration disease is selected from uveitis, vitritis, dry eye disease (DED), macular degeneration, idiopathic orbital inflammatory disease (IOD), chorioretinal inflammation, keratitis and combinations thereof.

3. The composition of claim 1, further comprising from about 0.01% w/ to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

4. The composition of claim 3, wherein said at least one additional active agent is selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof.

5. A dosage form comprising the composition of claim 1, wherein the composition is formulated in a dosage form selected from an ointment, a suspension, a cream, a spray, a lotion, a gel, an emulsion, a solution, an elixir, a tincture, a paste, a foam and drops.

6. The dosage form of claim 5, wherein the solution comprises nanomicelles comprising tapinarof.

7. A method of treatment, prevention and/or alleviation of an ocular inflammatory disease or ocular degeneration disease, comprising topically administering to the eyes of a subject in need thereof a therapeutically effective amount of a composition of claim 1, wherein the composition is formulated as an ophthalmic ointment, suspension, solution, drops, cream or foam.

8. The method of claim 7, wherein the ocular inflammatory disease or the ocular degeneration disease is selected from uveitis, vitritis, dry eye disease (DED), macular degeneration, idiopathic orbital inflammatory disease (IOD), chorioretinal inflammation, keratitis and combinations thereof.

9. The method of claim 7, wherein the treatment comprises once daily or twice daily.

10. The method of claim 18, wherein the treatment comprises once daily or twice daily topical administration to the eyes of a subject in need thereof.

11. The method of claim 18, wherein tapinarof and the at least one additional active agent exhibit an additive or synergistic effect, thereby allowing to reduce the amounts of the active agents in the composition.

12. A regimen of administration comprising the once daily or twice daily administration to the eyes of a patient in need thereof of a therapeutically effective amount of the composition of claim 1 until remission or alleviation of the ocular inflammatory disease or the ocular degeneration disease symptoms.

13. A regimen of administration comprising the once daily or twice daily administration to a patient in need thereof a therapeutically effective amount of the composition of claim 3 until remission or alleviation of the ocular inflammatory disease or the ocular degeneration disease symptoms.

14. The regimen of claim 12, wherein the ocular inflammatory disease or the ocular degeneration disease is selected from uveitis, vitritis, dry eye disease (DED), macular degeneration, idiopathic orbital inflammatory disease (IOD), chorioretinal inflammation, keratitis and combinations thereof.

15. A kit comprising one or more dosage forms of claim 5 and instructions for use.

16. A dosage form comprising the composition of claim 3, wherein the composition is formulated in a dosage form selected from an ointment, a suspension, a cream, a spray, a lotion, a gel, an emulsion, a solution, an elixir, a tincture, a paste, a foam and drops.

17. The dosage form of claim 16, wherein the solution comprises nanomicelles comprising tapinarof.

18. A method of treatment, prevention and/or alleviation of an ocular inflammatory disease or ocular degeneration disease, comprising topically administering to the eyes of a subject in need thereof a therapeutically effective amount of a composition of claim 3, wherein the composition is formulated as an ophthalmic ointment, suspension, solution, drops, cream or foam.

19. The method of claim 18, wherein the ocular inflammatory disease or the ocular degeneration disease is selected from uveitis, vitritis, dry eye disease (DED), macular degeneration, idiopathic orbital inflammatory disease (IOD), chorioretinal inflammation, keratitis and combinations thereof.

20. A regimen of administration comprising the once daily or twice daily administration to a patient in need thereof a therapeutically effective amount of the composition of claim 4 until remission or alleviation of the ocular inflammatory disease or the ocular degeneration disease symptoms.

21. The regimen of claim 13, wherein the ocular inflammatory disease or the ocular degeneration disease is selected from uveitis, vitritis, dry eye disease (DED), macular degeneration, idiopathic orbital inflammatory disease (IOD), chorioretinal inflammation, keratitis and combinations thereof.

22. A kit comprising one or more dosage forms of claim 16 and instructions for use.
